(19) United States
(12) Patent Application Publication
(10) Pub. No.: US 2025/0186634 A1
(43) Pub. Date: Jun. 12, 2025

(54) METHODS OF TREATING SMALL CELL LUNG CANCER

(71) Applicant: Evergreen Discovery, LLC., Springfield, NJ (US)

(72) Inventors: Giacomo Pirovano, Turin (IT); Thomas Lindner, New Brunswick, NJ (US); Martin Dolan, Morristown, NJ (US); Matthew Koslow, Lawrenceville, NJ (US); Thomas Reiner, Basel (CH)

(73) Assignee: Evergreen Discovery, LLC, Springfield, NJ (US)

(21) Appl. No.: 18/956,112

(22) Filed: Nov. 22, 2024

Related U.S. Application Data
(60) Provisional application No. 63/607,276, filed on Dec. 7, 2023.

(51) Int. Cl. A61K 51/08; A61K 31/4045; A61K 31/5513

(52) U.S. Cl. CPC A61K 51/088; A61K 31/4045; A61K 31/5513; A61K 2121/00; A61K 2123/00

(57) ABSTRACT

Methods of treating or diagnosing a patient having a cancer that expresses CCK2 receptors, such as small cell lung cancer (“SCLC”), colorectal cancer (CRC), and medullary thyroid cancer (“MTC”), with a radiolabeled peptide that avoids or minimizes the undesired uptake and binding of the radionuclide in the stomach of the patient.

### CROSS REFERENCE TO RELATED APPLICATIONS

This application claims the benefit of U.S. Provisional Application Ser. No. 63/607,276, filed on Dec. 7, 2023.

### FIELD OF THE INVENTION

The present invention is directed to a method of treating or diagnosing a patient having a cancer that expresses CCK2 receptors, such as, but not limited to, small cell lung cancer (“SCLC”) and medullary thyroid cancer (“MTC”), with a radiolabeled peptide that avoids or minimizes the undesired uptake and binding of the radionuclide in the stomach of the patient.

### BACKGROUND OF THE INVENTION

Cholecystokinin receptors are classified into two receptor subtypes, the cholecystokinin-1 (CCK1) receptor and the cholecystokinin-2 (CCK2) receptor. The CCK2 receptor is expressed in high incidence in a variety of cancers, including colorectal cancer (CRC), MTC, and SCLC. SCLC is a disease with a 7% 5-year survival rate, and presently, there is no cure. Approximately 30,000 people in the USA are diagnosed with SCLC on an annual basis. The CCK2 receptor has been found in SCLC patients with a positivity rate of about 38% based on patient Immunoreactive scores (“IRS”), which is a measure of both intensity of expression and proportion of expression per cell. The CCK1 receptor is expressed in a more limited number of human tumors.

Various radiolabeled peptide probes that target the CCK2 receptor have been developed based on the endogenous ligands for the CCK2 receptor, cholecystokinin (CCK) and gastrin. The two peptides, CCK and gastrin, bind to the CCK2 receptor with approximately the same affinity and potency and share a common bioactive region at the C-terminus, Trp-Met-Asp-Phe (M. Dufresne et al., Cholecystokinin and Gastrin Receptors, Physiol. Rev., 86:805-847, 2006), which has been shown to be essential for binding to the receptor (H. J. Tracy et al., The antral Hormone Gastrin: Physiological Properties of a Series of Synthetic Peptides structurally related to Gastrin I, Nature, 204:935-938, 1964). However, due to the very short physiological half-life of the parent peptide, synthetic modifications of the peptide that metabolically stabilize the peptide are generally required for medical application (M. Fani et al., Radiolabeled Peptides: Valuable Tools for the Detection and Treatment of Cancer, Theranostics, 2012, 2:481-501). In addition to radioligands based on modified peptides, non-peptidic radioligands have also been proposed (C. Wayua and P. S. Low, Evaluation of a Nonpeptidic Ligand for Imaging of Cholecystokinin 2 Receptor—Expressing Cancers, J. Nucl. Med., 56:113-119, 2015).

Targeting the CCK2 receptor offers a diagnostic and therapeutic approach to treating cancers that overexpress the CCK2 receptor, such as CRC, SCLC, and MTC. A variety of radiolabeled CCK2/gastrin-related peptides have been synthesized and characterized for imaging and/or treatment of tumors overexpressing CCK2 receptors (S. Roosenburg et al., Radiolabeled CCK/gastrin peptides for imaging and therapy of CCK2 receptor-expressing tumors, Amino Acids, 41:1049-1058, 2011). In therapeutic approaches, binding of the radiolabeled peptide to CCK2 receptors on the tumor puts the radioactive metal close to the tumor so that radiation emitted by the radionuclide damages DNA in the tumor causing the tumor to shrink.

In addition to being highly expressed in a variety of tumors, the CCK2 receptor is also expressed in high incidence in the stomach. Thus, probes that bind to CCK2 receptors on tumors can also bind to CCK2 receptors in the stomach. The CCK2 receptor in the stomach is responsible for mediating various hormones in the gut, namely gastrin and CCK. Although there are drugs for treating SCLC in various phases of clinical development, very few of these drugs contain a radiolabeled peptide as a therapeutic and/or diagnostic (theragnostic) agent. Lutetium-177 (¹⁷⁷Lu) and Gallium-68 (⁶⁸Ga) are two radioisotopes that have therapeutic and diagnostic applications, respectively, that can potentially be used to treat SCLC. Administering a radiolabeled peptide to target CCKR2, however, is often associated with an undesired uptake and binding of the radionuclide in the stomach of the patient. This reduces the therapeutic window by binding to CCK2 receptors in an off-target tissue (i.e., stomach vs tumor) making treatments less effective.

There is a need in the art of treating a patient with tumor that expresses the CCK2 receptor, such as CRC, SCLC, and MTC, with a radiolabeled peptide that avoids or minimizes the undesired uptake and binding of the radionuclide in the stomach of the patient. The inventive method described herein addresses this unmet need.

### SUMMARY OF THE INVENTION

The invention is directed to a method of diagnosing if a patient has a tumor that expresses CCK2 receptors or treating a patient with a tumor that expresses CCK2 receptors comprising (i) orally administering to the patient a blocking agent and (ii) subsequently intravenously or topically administering to the patient a radiopharmaceutical of formula:

Z-L-X-X1-X2-Asp-X3

wherein
Z is a chelator capable of chelating a radioactive isotope,
L is absent or is a linker,
X is absent or is an amino acid sequence, and
X1, X2, and X3 are lipophilic amino acids, and
   wherein a radioactive isotope is complexed by the chelator.

In one embodiment, the radioactive isotope is selected from the group consisting of ¹⁷⁷Lu and ⁶⁸Ga. In one embodiment, the radioactive isotope is ¹⁷⁷Lu. In one embodiment, the radioactive isotope is ⁶⁸Ga.

In one embodiment, the radiopharmaceutical is administered intravenously. In one embodiment, the radiopharmaceutical is administered topically.

In one embodiment, the tumor that expresses CCK2 receptors is SCLC or MTC. In one embodiment, the tumor that expresses CCK2 receptors is MTC. In one embodiment, the tumor that expresses CCK2 receptors is SCLC.

In one embodiment, the invention is directed to a method of diagnosing if a patient has a tumor that expresses CCK2 receptors or treating a patient with a tumor that expresses CCK2 receptors comprising (i) orally administering to the patient a blocking agent and (ii) subsequently intravenously or topically administering to the patient a radiopharmaceutical of formula:

DOTA-D-Glu-Ala-Tyr-Gly-Trp-(N-Me)Nle-Asp-1-Nal-NH_{2 (i.e., EVG}321)

wherein a radioactive isotope is complexed by the chelator.

In one embodiment, the invention is directed to a method of diagnosing if a patient has a tumor that expresses CCK2 receptors or treating a patient with a tumor that expresses CCK2 receptors comprising (i) orally administering to the patient a blocking agent and (ii) subsequently intravenously or topically administering to the patient a radiopharmaceutical of formula:

DOTA-DGlu-DGlu-DGlu-DGlu-DGlu-DGlu-Ala-Tyr-Gly-Trp-Met-Asp-Phe-NH₂ (i.e., DOTA-PP-F11)

wherein a radioactive isotope is complexed by the chelator.

In one embodiment, the invention is directed to a method of diagnosing if a patient has a tumor that expresses CCK2 receptors comprising (i) orally administering to the patient a blocking agent and (ii) subsequently intravenously or topically administering to the patient a pharmaceutical of formula:

Fl-DGlu-DGlu-DGlu-DGlu-DGlu-DGlu-Ala-Tyr-Gly-Trp-Met-Asp-Phe-NH_{2 (F}1-PP-F11)

wherein Fl is a fluorophore.

In one embodiment, the fluorophore is FITC, i.e.:

FITC-DGlu-DGlu-DGlu-DGlu-DGlu-DGlu-Ala-Tyr-Gly-Trp-Met-Asp-Phe-NH₂ (FITC-PP-F11).

wherein FITC is fluorescein isothiocyanate.

In one embodiment, the radioactive isotope is selected from the group consisting of ¹⁷⁷Lu and ⁶⁸Ga. In one embodiment, the radioactive isotope is ¹⁷⁷Lu. In one embodiment, the radioactive isotope is ⁶⁸Ga.

In one embodiment, the radiopharmaceutical is administered intravenously. In one embodiment, the radiopharmaceutical is administered topically.

In one embodiment, the tumor that expresses CCK2 receptors is SCLC or MTC. In one embodiment, the tumor that expresses the CCK2 receptor is MTC. In one embodiment, the tumor that expresses the CCK2 receptor is SCLC.

### BRIEF DESCRIPTION OF THE DRAWINGS

FIG. 1 illustrates small molecule antagonism of fluorescently labelled DOTA-PP-F11 (PP-F11-FITC or FITC-PP-F11) in AR42J and Panc1 cell lines. Results were measured by measuring the intensity of the fluorescent (“FL”) signal of FL tagged PP-F11 in the presence or absence of antagonist small molecule inhibitors. FIG. 1A represents experiments in AR42J cells and FIG. 1B represents experiments in Panc1 cells. Each column represents a specific experimental condition. Specifically, A=PP-F11-FITC, B=PP-F11-FITC+CI988, C=PP-F11-FITC+Proglumide, D=PP-F11-FITC+netrazepide, and E=PP-F11-FITC+PP-F11 (excess dose).

FIG. 2 graphically depicts the proposed mechanism by which the method is believed to work, i.e., by blocking of EVG321 to receptors in the stomach.

FIG. 3 compares the uptake of ¹⁷⁷Lu-EVG321 by AR42J rat pancreatic cancer cell lines cultured in vitro that have been exposed to the blocking agents CI-988, proglumide, and sograzepide with cells that have not been exposed to a blocking agent as described in Example 2.

### DETAILED DESCRIPTION OF THE INVENTION

The invention is directed to a method of diagnosing if a patient has a tumor that expresses CCK2 receptors or treating a patient with a tumor that expresses CCK2 receptors. The method comprises (i) orally administering to the patient a blocking agent and (ii) subsequently intravenously or topically administering to the patient a radiopharmaceutical, wherein the radiopharmaceutical comprises an amino acid sequence that binds to CCK2 receptors and a chelator capable of complexing a radioactive isotope, and wherein a radioactive isotope is complexed by the chelator.

In one embodiment, the method of diagnosing if a patient has a tumor that expresses CCK2 receptors or treating a patient with a tumor that expresses CCK2 receptors comprises (i) orally administering to the patient a blocking agent and (ii) subsequently intravenously or topically administering to the patient a radiopharmaceutical of formula:

Z-L-X-X1-X2-Asp-X3

wherein:
Z is a chelator capable of complexing a radioactive isotope,
L is absent or is a linker,
X is absent or is an amino acid sequence,
X1, X2, and X3 are each a lipophilic amino acid, and
   wherein a radioactive isotope is complexed by the chelator.

### Definitions

The term “diagnosing,” as used herein, means detecting if a patient has a tumor that expresses CCK2 receptors based on established methods of diagnosis such as computer tomography (CT), magnetic resonance (MRI), scintigraphy, Single Photon Emission Computet Tomography (SPECT), Positron Emission Tomorgraphy (PET), or other similar technique including any diagnostic medical exam or evaluation.

The term “treating,” as used herein, means reducing the size of a tumor that expresses CCK2 receptors in a patient or stalling growth of a tumor (stasis).

The term “patient,” as used herein, means a mammal. In one embodiment, the patient is a human.

The term “linker,” as used herein, means a chemical moiety that connects two individual chemical moieties. The terms “linker” and “spacer” are used interchangeably in the literature to describe such a chemical moiety. The linker can be bound to the N-terminus of the amino acid polymer of the radiopharmaceutical (i.e., X-X1-X2-Asp-X3), integrated within the amino acid polymer, or conjugated to a side chain of the amino acid sequence, and is used as a separator between the amino acid polymer of the radiopharmaceutical and the chelator.

The linker may be any chemical moiety, such as polyethylene glycol (“PEG”), a carbohydrate, or an amino acid, as well as an aminohexanoyl, aminobenzoyl, or piperidine moiety. Typically, the linker is a carbon-containing chain of 1 to 20, preferably 4 to 15, and more preferably 1 to 4 atoms in length. The linker can also be cyclic, such as a TRAP chelator core, and be substituted with functional groups such as carboxylic acids, phenols, or amines to improve target binding, pharmacokinetics, and/or pharmacodynamics.

The phrase “chelator,” as used herein, means a molecule with functional groups, such as amine or carboxylic groups, that is capable of complexing the radioactive isotope via non-covalent bonds. Suitable chelators include, but are not limited to, derivatives of 1,4,7,10-tetraazacylododecane, 1,4,7-triazacyclononane, 1,4,8,11-tetraazacyclotetradecane, diethylenetriamine, 3,6,9,15-tetraazabicyclo[9.3.1]pentadeca-1 (15),11,13-triene, and 6-aminoperhydro-1,4-diazepine. Illustrative chelators include, but are not limited to, DOTA (1,4,7,10-tetraazacyclododecanetetraacetic acid); DOTAGA (2-[1,4,7,10-tetraazacyclododecane-4,7,10-tris(acetic acid)]-pentanedioic acid); p-SCN-Bn-DOTA (S-2-(4-Isothiocyanatobenzyl)-1,4,7,10-tetraazacyclododecane tetraacetic acid); DOTAM (1,4,7,10-Tetraazacyclododecane-1,4,7,10-tetracarbamoylmethyl); EDTA (ethylenediaminetetraacetic acid); DTPA (diethylenetriaminepentaacetic acid); ITC-MX (1-p-Isothiocyanato-benzyl-methyl-diethylenetriaminepentaacetic acid); NOTA (1,4,7-triazacyclononane-1,4,7-triacetic acid), 1,4,7-triazacyclononane,1-glutaric acid-4,7-acetic acid); DTPA (diethylenetriaminepentaacetic acid); BAPTA (1,2-bis(o-aminophenoxy) ethane-N,N,N′,N′-tetraacetic acid)); TETA (1,4,8,11-tetraazacyclotetradecane-1,4,8,11-tetraacetic acid), or a derivative thereof, such CB-TE2A (4,11-bis-(carboxymethyl)-1,4,8,11-tetraazabicyclo[6.6.2]-hexadecane); SarAr (1-N-(4-aminobenzyl)-3,6,10,13,16,19-hexaazabicyclo[6.6.6]-eicosane-1,8-diamine; TRAP (1,4,7-triazacyclononane-1,4,7-tris [methyl (2-carboxyethyl)phosphinic acid); HBED (N, NO-bis(2-hydroxybenzyl)-ethylenediamine-N, NO-diacetic acid); 2,3-EIOPO (3-hydroxypyridin-2-one); PCTA (3,6,9,15-tetraazabicyclo[9.3.1]-pentadeca-1 (15),11,13-triene-3,6,9-triacetic acid); OCTAPA (N, NO-bis(6-carboxy-2-pyridylmethyl)-ethylenediamine-N, NO-diacetic acid); and H2-MACROPA (N,N′-bis[(6-carboxy-2-pyridil)methyl]-4,13-diaza-18-crown-6); THP (N,N′,N″-tris-[1-methyl-2-aminomethyl-3-hydroxy-4-pyridinone]-3-aminotrispropionate); PCTA (3,6,9,15-tetraazabicyclo[9.3.1]pentadeca-1 (15),11,13-triene-3,6,9-triacetic acid; AAZTA (6-amino-6-methylperhydro-1,4-diazepinetetraacetic acid); DATA ((6-pentanoic acid)-6-(amino) methy-1,4-diazepinetriacetate); and DFO (desferrioxamine-B). Preferred chelators include derivatives of 1,4,7,10-tetraazacyclododecan such as DOTA, DOTAM, DOTAGA, and p-SCN-Bn-DOTA. Illustrative chelators include, but are not limited to, those described in, for example, U.S. Pat. No. 10,874,753; US 2022/0339304; WO 2023/191839, US 2023/0348553, and WO 2023/201435, the contents of which are expressly incorporated herein. A preferred chelator is DOTA.

The phrase, “amino acid,” as used herein, has its ordinary meaning in the art, i.e., a compound that includes a central carbon atom attached to a hydrogen, an acidic carboxyl group (—COOH), an amino group (—NH₂), and an organic side chain (often called an R group). Amino acids include both D- and L-amino acids. The stereochemistry of naturally occurring amino acids are the L-amino acids.

The phrase “lipophilic amino acid,” as used herein, means an amino acid with an alkyl or aromatic side chain that has a logP value higher than that of alanine. Illustrative lipophilic natural amino acids include glycine (Gly), alanine (Ala), valine (Val), leucine (Leu), isoleucine (Ile), proline (Pro), phenylalanine (Phe), methionine (Met), and tryptophan (Trp). Illustrative lipophilic unnatural amino acids include, but are not limited to, beta-alanine; 1-naphthylalanine (1Nal); 2-naphthylalanine (2Nal); tert-butylglycine, 3-(1-adamantyl)-2-aminopropanoic acid; cyclohexylalanine; cyclohexylglycine; norleucine (Nle); and their N-methylated derivatives, such as N-methyl norleucine ((N-Me)-Nle); as well as their alpha methylated derivatives such as alpha-methyl-tryptophane (aMe-Trp).

The phrase natural amino acid,” as used herein, means one of the twenty two naturally occurring amino acids, i.e., aspartic acid (Asp), tyrosine (Tyr), leucine (Leu), tryptophan (Trp), arginine (Arg), valine (Val), glutamic acid (Glu), methionine (Met), phenylalanine (Phe), serine (Ser), alanine (Ala), glutamine (Gln), glycine (Gly), proline (Pro), threonine (Thr), asparagine (Asn), lysine (Lys), histidine (His), isoleucine (Ile), cysteine (Cys), selenocysteine (Sec), and pyrrolysine (Pyl).

The phrase “unnatural amino acid,” as used herein, means an amino acid other than one of the 22 naturally occurring amino acids. Unnatural amino acids include any amino acid, modified amino acid, and/or an analog thereof, that is not one of the natural amino acids. Illustrative unnatural amino acids include, but are not limited to diaminopropionic acid (Dap), diaminobutyric acid (Dab), ornithine (Orn), aminoadipic acid, β-alanine, α-aminohexanoic acid (Ahx), 6-aminohexanoic acid, 8-aminooctanoic acid, 1-naphthylalanine (1Nal), 2-naphthylalanine (2Nal), 3-(1-naphthyl) alanine, 3-(2-naphthyl) alanine, 4-(aminomethyl)cyclohexane carboxylic acid (Amc), γ-aminobutiric acid (GABA), 3-(aminomethyl)benzoic acid, p-ethynyl-phenylalanine, p-propargly-oxy-phenylalanine, m-ethynyl-phenylalanine, p-bromophenylalanine, p-iodophenylalanine, p-azidophenylalanine, p-acetylphenylalanine, azidonorleucine, 6-ethynyl-tryptophan, 5-ethynyl-tryptophan, 3-(6-chloroindolyl) alanine, 3-(6-bromoindolyl) alanine, 3-(5-bromoindolyl) alanine, azidohomoalanine, p-chlorophenylalanine, α-aminocaprylic acid, O-methyl-L-tyrosine, N-acetylgalactosamine-α-threonine, N-acetylgalactosamine-α-serine, norleucine (Nle), and N-methyl norleucine ((N-Me)-Nle). The phrase “unnatural amino acid” is also used for natural amino acids which are methylated or otherwise substituted at any position.

The phrase “blocking agent,” as used herein, means a compound that is an antagonist that binds to the CCK2 receptor and does not contain a radioactive element other than naturally occurring radioactive isotopes.

The term “fluorophore,” as used herein means a fluorescent chemical compound that can re-emit light upon light excitation.

The Method of Treating or Diagnosing a Patient with a Tumor that Expresses CCK2 Receptors

The method of diagnosing if a patient has a tumor that expresses CCK2 receptors or treating a patient with a tumor that expresses CCK2 receptors comprises (i) orally administering to the patient a blocking agent and (ii) subsequently intravenously or topically administering to the patient a radiopharmaceutical, wherein the radiopharmaceutical comprises an amino acid sequence that binds to CCK2 receptors that is covalently attached to a chelator capable of complexing a radioactive isotope, and wherein a radioactive isotope is complexed by the chelator.

Any radiopharmaceutical that includes an amino acid sequence that binds to CCK2 receptors can be used in the method.

In one embodiment, the method comprises (i) orally administering to the patient a blocking agent and (ii) subsequently intravenously or topically administering to the patient a radiopharmaceutical of formula:

Z-L-X-X1-X2-Asp-X3

wherein
Z is a chelator capable of complexing a radioactive isotope,
L is absent or is a linker,
X is absent or is an amino acid sequence, and
X1, X2, and X3 are each a lipophilic amino acid, and
   wherein a radioactive isotope is complexed by the chelator.

In one embodiment, X is an amino acid sequence ranging from 1 to 20 amino acids, preferably from 1 to 10 amino acids, and more preferably from 1 to 5 amino acids. The amino acids in the amino acid sequence X can be a natural amino acid or an unnatural amino acid.

X1 is a lipophilic amino acids. X1 can be a natural or unnatural amino. X1 is preferably an aromatic amino acid. An illustrative amino acid for X1 is tryptophane.

X2 is a lipophilic amino acid. X2 can be a natural or unnatural amino acid. X2 is preferably an aliphatic amino acid, such as methionine, cyclohexylglycine, and norleucine. In a preferred embodiment, X2 is substituted with a methyl group on its alpha-nitrogen atom. In one embodiment, X2 is N-methyl norleucine.

X3 is a lipophilic amino acid. X3 can be an aromatic or aliphatic amino acid and can be a natural or unnatural amino acid. Illustrative amino acids for X3 include, but are not limited to phenylalanine, N-methylphenylalanine, 1-adamantylalanine, 1-naphtylalanine, and 2-napthylamine.

Radioactive isotopes that can be complexed by the chelator include, but are not limited to, α-, β-, and/or γ-emitters. The radioactive isotopes can be used in diagnostic and/or therapeutic applications. Illustrative radioactive isotopes include, but are not limited, to ¹⁷⁷Lu, ⁶⁸Ga, ⁶⁷Ga, ^{99m}Tc, ¹⁸⁸Re, ¹⁸⁶Re, ¹⁵³Sm, ¹¹¹In, ⁵⁹Fe, ⁶³Zn, ⁵²Fe, ⁴⁵Ti, ⁶⁰Cu, ⁶¹Cu, ⁶⁷Cu, ⁶⁴Cu, ⁶²Cu, ¹⁹⁸Au, ¹⁹⁹Au, ^{195m}Pt, ^{191m}Pt, ^{193m}Pt, ¹⁹⁷Pt, ^{117m}Sn, ⁸⁹Zr, ¹⁸F, and ¹²⁴I, ¹²⁵I, ¹³¹I, ⁹⁰Y, ⁸⁹Sr, ¹¹¹In, ¹⁵³Gd, ²²⁵Ac, ²¹²Bi, ²¹³Bi, ²¹¹At, ^{117m}Sn, ¹⁰³Pd, ^{103m}Rh, ²²³Ra, ²²⁴Ra, ²²⁷Th, ³²P, ¹⁴⁹Tb, ¹⁴⁷Th, ²¹²Pb, ²⁰³Pb, ¹⁶¹Tb, ³³P, ²⁰¹Tl, ¹¹⁹Sb, ^{58m}Co, ¹⁶¹Ho, ²⁰³Pb, ⁷⁴Br, ⁷⁵Br, ⁷⁶Br, ⁷⁷Br, ⁸⁰Br, ⁸²Br, ⁸⁵Br. Preferred radioactive isotopes are ¹⁷⁷Lu and ⁶⁸Ga.

A person of ordinary skill in the art would readily understand what radioactive isotopes are useful for diagnosing if a patient has a tumor that expresses CCK2 receptors and what radioactive isotopes are useful for treating a patient who has a tumor that expresses CCK2 receptors.

Illustrative radioactive isotopes for treating a patient include, but are not limited to, ¹⁸⁸Re, ¹⁸⁶Re, ¹⁵³Sm, ¹⁶⁶Ho, ⁹⁰Y, ⁸⁹Sr, ¹¹¹In, ¹⁵³Gd, ²²⁵Ac, ²¹²Bi, ²¹³Bi, ²¹¹At, ⁶⁰Cu, ⁶¹Cu, ⁶⁷Cu, ⁶⁴Cu, ⁶²Cu, ¹⁹⁸Au, ¹⁹⁹Au, ^{195m}Pt, ^{193m}Pt, ¹⁹⁷Pt, ^{117m}Sn, ¹⁰³Pd, ²¹²Pb, ^{103m}Rh, ¹⁷⁷Lu, ²²³Ra, ²²⁴Ra, ²²⁷Th, ³²P, ¹⁶¹Tb, ³³P, ¹²⁴I, ¹²⁵I, ¹³¹I, ²⁰³Pb, ²⁰¹Tl, ¹¹⁹Sb, ^{58m}Co, ⁷⁴Br, ⁷⁵Br, ⁷⁶Br, ⁷⁷Br, ⁸⁰Br, ⁸²Br, ⁸⁵Br, and ¹⁶¹Ho.

Illustrative radioactive isotopes for diagnosing a patient include, but are not limited to, ^{99m}Tc, ¹⁸⁸Re, ¹⁸⁶Re, ¹⁵³Sm, ⁶⁷Ga, ⁶⁸Ga, ¹¹¹In, ⁵⁹Fe, ⁶³Zn, ⁵²Fe, ⁴⁵Ti, ⁶⁰Cu, ⁶¹Cu, ⁶⁷Cu, ⁶⁴Cu, ⁶²Cu, ¹⁹⁸Au, ¹⁹⁹Au, ^{195m}Pt, ^{191m}Pt, ^{193m}Pt, ^{117m}Sn, ⁸⁹Zr, ¹⁷⁷Lu, ¹⁴⁷Tb, ²¹²Pb, ²⁰³Pb, ¹⁸F, ⁷⁴Br, ⁷⁵Br, ⁷⁶Br, ⁷⁷Br, ⁸⁰Br, ⁸²Br, ⁸⁵Br and ¹²³I.

When the method is used for diagnosing if a patient has a tumor that expresses CCK2 receptors the preferred radioisotope is 68Ga. When the method is used for treating a tumor that expresses CCK2 receptors the preferred radioisotope is ¹⁷⁷Lu.

The blocking agent has a high affinity for binding to CCK2 receptors in the stomach. The blocking agent can be any selective CCK2 receptor inhibitor that is orally administrable. Suitable blocking agents include, but are not limited to, sograzepide, proglumide, lorglumide, devazepide, CI-988, CI-1015, L-365,260, L-369,293, RP-69758, LY-288,513, PD-145,942, and the CCKB receptor inverse agonists L-740,093, PF-04756956, JNJ-26070109. Preferably, the blocking agent is sograzepide.

The structure of sograzepide ((R)-1-(1-(3,3-dimethyl-2-oxobutyl)-2-oxo-5-(pyridin-2-yl)-2,3-dihydro-1H-benzo[e][1,4]diazepin-3-yl)-3-(3-(methylamino)phenyl) urea), also known as netazepide, is:

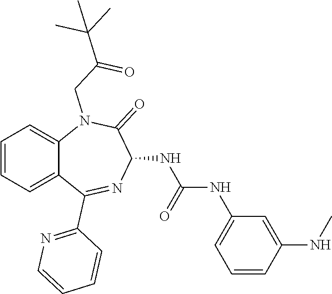 [SMILES: CNc1cccc(NC(=O)N[C@@H]2N=C(c3ccccn3)c3ccccc3N(CC(=O)C(C)(C)C)C2=O)c1]

The structure of CI-988 (4-([(1R)-2-([(2R)-3-(1H-indol-3-yl)-2-methyl-1-oxo-2-([(tricyclo[3.3.1.13,7]dec-2-yloxy) carbonyl]amino)propyl]amino)-1-phenylethyl]amino)-4-oxobutanoic acid) is:

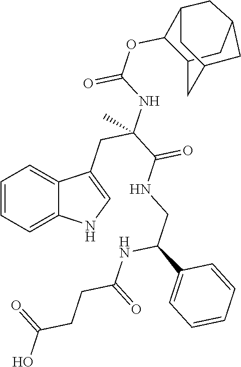 [SMILES: C[C@](Cc1c[nH]c2ccccc12)(NC(=O)OC1C2CC3CC(C2)CC1C3)C(=O)NC[C@H](NC(=O)CCC(=O)O)c1ccccc1]

The structure of proglumide (4-benzamido-5-(dipropylamino)-5-oxopentanoic acid) is:

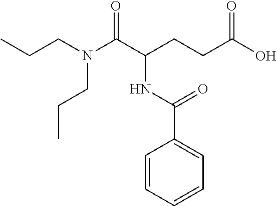 [SMILES: CCCN(CCC)C(=O)C(CCC(=O)O)NC(=O)c1ccccc1]

The structure of lorglumide (4-[(3,4-dichlorobenzoyl)amino]-5-(dipentylamino)-5-oxopentanoic acid) is:

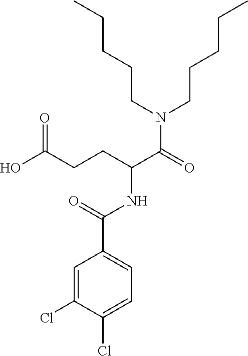 [SMILES: CCCCCN(CCCCC)C(=O)C(CCC(=O)O)NC(=O)c1ccc(Cl)c(Cl)c1]

The structure of devazepide (N-[(3S)-1-methyl-2-oxo-5-phenyl-3H-1,4-benzodiazepin-3-yl]-1H-indole-2-carboxamide) is:

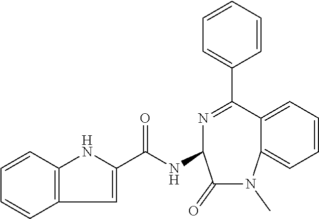 [SMILES: CN1C(=O)[C@@H](NC(=O)c2cc3ccccc3[nH]2)N=C(c2ccccc2)c2ccccc21]

The structure of CI-1015 (2-adamantyl N-[(2R)-1-[[(1S,2S)-2-hydroxycyclohexyl]amino]-3-(1H-indol-3-yl)-2-methyl-1-oxopropan-2-yl]carbamate) is

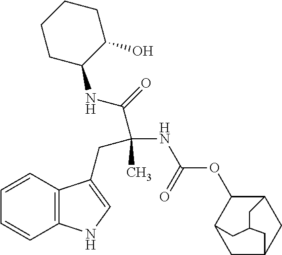 [SMILES: C[C@](Cc1c[nH]c2ccccc12)(NC(=O)OC1C2CC3CC(C2)CC1C3)C(=O)N[C@H]1CCCC[C@@H]1O]

The structure of L-365,260 (N-[(3R)-2,3-Dihydro-1-methyl-2-oxo-5-phenyl-1H-1,4-benzodiazepin-3-yl]-N′-(3-methylphenyl) urea) is:

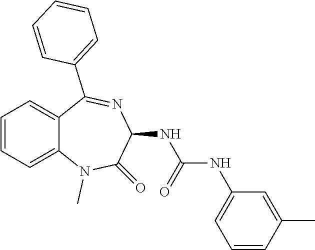 [SMILES: Cc1cccc(NC(=O)N[C@@H]2N=C(c3ccccc3)c3ccccc3N(C)C2=O)c1]

The structure of L-369,293 (N-[(3R)-2,3-dihydro-1-methyl-2-oxo-5-phenyl-1H-1,4-benzodiazepin-3-yl]-N′-(3-methylphenyl)-urea) is:

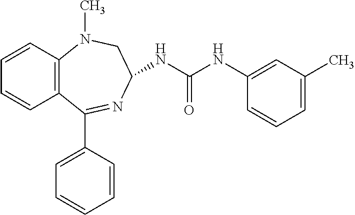 [SMILES: Cc1cccc(NC(=O)N[C@H]2CN(C)c3ccccc3C(c3ccccc3)=N2)c1]

The structure of RP-69758 (2-[3-[[2-[[2-(methyl-phenylamino)-2-oxoethyl]-phenylamino]-2-oxoethyl]carbamoylamino]phenyl]acetic acid) is:

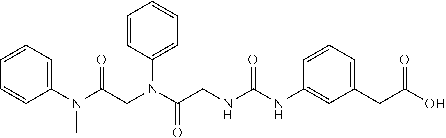 [SMILES: CN(C(=O)CN(C(=O)CNC(=O)Nc1cccc(CC(=O)O)c1)c1ccccc1)c1ccccc1]

The structure of LY-288,513 ((4S,5R)—N-(4-bromophenyl)-3-oxo-4,5-diphenylpyrazolidine-1-carboxamide) is:

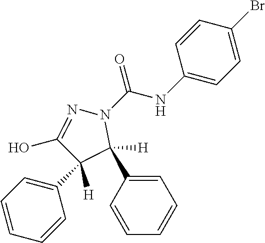 [SMILES: O=C(Nc1ccc(Br)cc1)N1N=C(O)[C@@H](c2ccccc2)[C@@H]1c1ccccc1]

The structure of PD-145,942 ([1-(2-Hydroxy-cyclohexylcarbamoyl)-2-(1H-indol-3-yl)-1methyl-ethyl]carbamic acid adamantan-2-yl ester) is:

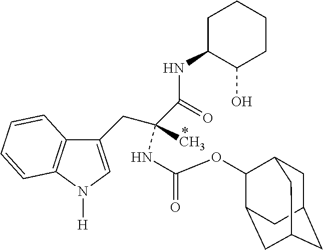 [SMILES: C[C@](Cc1c[nH]c2ccccc12)(NC(=O)OC1C2CC3CC(C2)CC1C3)C(=O)N[C@H]1CCCC[C@@H]1O]

The structure of L-740,093 (1-[(3R)-5-(3-azabicyclo[3.2.2]nonan-3-yl)-1-methyl-2-oxo-3H-1,4-benzodiazepin-3-yl]-3-(3-methylphenyl) urea) is:

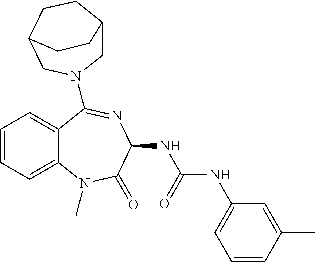 [SMILES: Cc1cccc(NC(=O)N[C@@H]2N=C(N3CC4CCC(CC4)C3)c3ccccc3N(C)C2=O)c1]

The structure of PF-04756956 ((4S)-4-((1H-indazol-3-yl)methyl)-6-(2-((R)-2-(3,5-dimethylbenzyl)piperidin-1-yl)-2-oxoethyl)-1-phenyl-4H-benzo[b][1,2,4]triazolo[4,3-d][1,4]diazepin-5 (6H)-one) is:

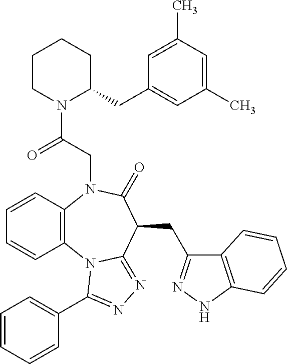 [SMILES: Cc1cc(C)cc(C[C@H]2CCCCN2C(=O)CN2C(=O)[C@@H](Cc3n[nH]c4ccccc34)c3nnc(-c4ccccc4)n3-c3ccccc32)c1]

The structure of JNJ-26070109 ((R) 4-bromo-N-[1-(2,4-difluoro-phenyl)-ethyl]-2-(quinoxaline-5-sulfonylamino)-benzamide) is:

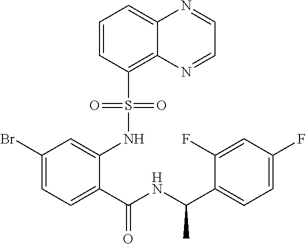 [SMILES: C[C@@H](NC(=O)c1ccc(Br)cc1NS(=O)(=O)c1cccc2nccnc12)c1ccc(F)cc1F]

The blocking agent is typically orally administered at a dose ranging from about 5 mg to about 1,000 mg, preferably about 5 mg to about 500 mg, more preferably about 5 mg to about 250 mg.

The blocking agent sograzepide is typically orally administered at a dose ranging from about 5 mg to about 500 mg, preferably from about 10 mg to about 350 mg, and more preferably from about 20 to about 250 mg.

The blocking agent CI988 is typically orally administered at a dose ranging from about 5 to about 500 mg, preferably from about 5 mg to about 350 mg, and more preferably from about 5 to about 250 mg.

In one embodiment, the radiopharmaceutical is administered intravenously or topically following oral administration of the blocking agent. In one embodiment, the radiopharmaceutical is co-administered intravenously or topically with oral administration of the blocking agent.

In one embodiment, the blocking agent is administered between about 1 minute and about 180 minutes prior to intravenous or topical administration of the radiopharmaceutical. In one embodiment, the blocking agent is administered between about 5 minute and about 120 minutes prior to intravenous or topical administration of the radiopharmaceutical. In one embodiment, the blocking agent is administered between about 10 minute and about 60 minutes prior to intravenous or topical administration of the radiopharmaceutical. In one embodiment, the blocking agent is co-administered with the radiopharmaceutical.

The blocking agent may be administered in any form convenient for oral administration including, but not limited to, a tablet, a pill, a capsule, a caplet, a syrup, an elixir, a solution, a suspension, and an emulsion.

The radiopharmaceutical may be administered intravenously or topically in a dose that can allow diagnosis or therapy of target-expressing organs or malignances. Dosing regimens for administering radiopharmaceuticals are known in the art.

In one embodiment, X1 is Trp, X2 is (N-Me) Nle, X3 is 1-Nal-NH₂, L is D-Glu, and X is Ala-Tyr-Gly.

In one embodiment, X1 is Trp, X2 is (N-Me) Nle, and X3 is 1-Nal-NH₂, L is D-Glu, X is Ala-Tyr-Gly, and Z is DOTA.

In one embodiment, X1 is Trp, X2 is (N-Me) Nle, and X3 is 1-Nal-NH₂, L is D-Glu, X is Ala-Tyr-Gly, Z is DOTA, and the radioactive isotope is ¹⁷⁷Lu or ⁶⁸Ga.

In one embodiment, the radiopharmaceutical is Z-DGlu-PEG-Trp-(NMe) Ile-Asp-Nal. In one embodiment, Z is DOTA, i.e., DOTA-DGlu-PEG-Trp-(NMe) Ile-Asp-Nal. In one embodiment, the radiopharmaceutical is DOTA-DGlu-PEG-Trp-(NMe) Ile-Asp-Nal and the radioactive isotope is ¹⁷⁷Lu or ⁶⁸Ga.

In one embodiment, the radiopharmaceutical is Z-DGlu6-Ala-Tyr-GlyTrp-Met-Asp-Phe-NH₂. In one embodiment Z is DOTA, i.e., DOTA-DGlu6-Ala-Tyr-Gly-Trp-Met-Asp-Phe-NH₂ (referred to herein as DOTA-PP-F11). In one embodiment, the radiopharmaceutical is DOTA-DGlu6-Ala-Tyr-Gly-Trp-Met-Asp-Phe-NH₂ and the radioactive isotope is ¹⁷⁷Lu or ⁶⁸Ga.

In a preferred embodiment, the method of treating a patient with a tumor that expresses CCK2 receptors involves first orally administering to the patient a blocking agent and, following administration of the blocking agent, intravenously or topically administering to the patient a radiopharmaceutical of formula:

DOTA-D-Glu-Ala-Tyr-Gly-Trp-(N-Me)Nle-Asp-1-Nal-NH₂ (referred to herein as EVG321);

wherein a radioactive isotope is complexed by the DOTA. In a preferred embodiment, the radioactive isotope is complexed to ¹⁷⁷Lu.

In a preferred embodiment, the tumor that expresses CCK2 receptors is selected from the group consisting of SCLC and MTC. In one embodiment, the cancer is SCLC. In one embodiment, the cancer is MTC.

In a preferred embodiment, the method of diagnosing if a patient has a tumor that expresses CCK2 receptors involves first orally administering to the patient a blocking agent and, following administration of the blocking agent, intravenously or topically administering to the patient a radiopharmaceutical of formula:

DOTA-D-Glu-Ala-Tyr-Gly-Trp-(N-Me)Nle-Asp-1-Nal-NH₂ (i.e., EVG321);

wherein a radioactive isotope is complexed by the DOTA. In a preferred embodiment, the radioactive isotope is complexed to ⁶⁸Ga.

In a preferred embodiment, the tumor that expresses CCK2 receptors is selected from the group consisting of SCLC and MTC. In one embodiment, the cancer is SCLC. In one embodiment, the cancer is MTC.

The chemical structure of EVG321 is:

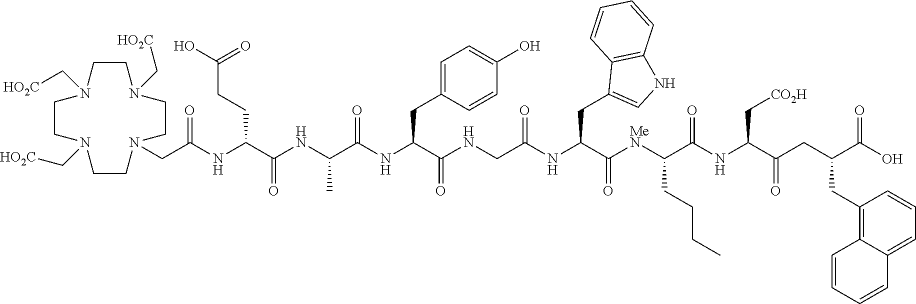 [SMILES: CCCC[C@@H](C(=O)N[C@@H](CC(=O)O)C(=O)C[C@@H](Cc1cccc2ccccc12)C(=O)O)N(C)C(=O)[C@H](Cc1c[nH]c2ccccc12)NC(=O)CNC(=O)[C@H](Cc1ccc(O)cc1)NC(=O)[C@H](C)NC(=O)[C@@H](CCC(=O)O)NC(=O)CN1CCN(CC(=O)O)CCN(CC(=O)O)CCN(CC(=O)O)CC1]

The chemical structure of DOTA-CCK-66 is:

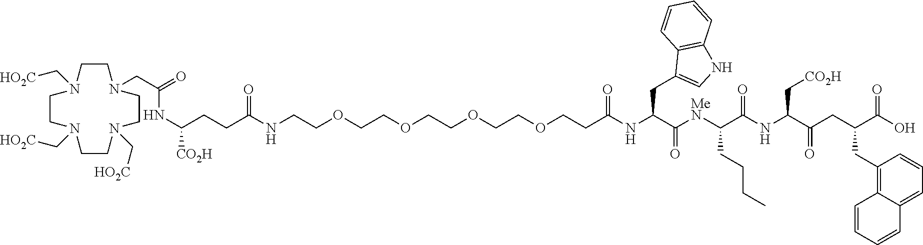 [SMILES: CCCC[C@@H](C(=O)N[C@@H](CC(=O)O)C(=O)C[C@@H](Cc1cccc2ccccc12)C(=O)O)N(C)C(=O)[C@H](Cc1c[nH]c2ccccc12)NC(=O)CCOCCOCCOCCOCCNC(=O)CC[C@@H](NC(=O)CN1CCN(CC(=O)O)CCN(CC(=O)O)CCN(CC(=O)O)CC1)C(=O)O]

The formula of PP-F11N is: DOTA-(DGlu) 6-Ala-Tyr-Gly-Trp-Nle-Asp-Phe-NH₂

In an embodiment, the method of diagnosing if a patient has a tumor that expresses CCK2 receptors comprises (i) orally administering to the patient a blocking agent and (ii) subsequently intravenously or topically administering to the patient a pharmaceutical of formula:

Fl-DGlu-DGlu-DGlu-DGlu-DGlu-DGlu-Ala-Tyr-Gly-Trp-Met-Asp-Phe-NH₂,

wherein Fl is a fluorophore.

Suitable fluorophores include, but are not limited to, FTIC, IR780, IR800, IR780, DY-684, DY-700, Janelia669, BODIPY, BODIPY665, sulfo-CY5, CY5.5, CY7, CY7.5, ICG, IR780, IR140, or DiR.5.

In one embodiment, the fluorophore is FTIC, i.e. the radiopharmaceutical is:

FITC-DGlu-DGlu-DGlu-DGlu-DGlu-DGlu-Ala-Tyr-Gly-Trp-Met-Asp-Phe-NH₂

### (FITC-PP-F11).

FITC is fluorescein isothiocyanate. FITC can be either 5- or 6-FITC. The structure of 5-FITC is:

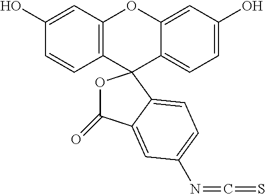 [SMILES: O=C1OC2(c3ccc(O)cc3Oc3cc(O)ccc32)c2ccc(N=C=S)cc21]

and the structure of 6-FITC is:

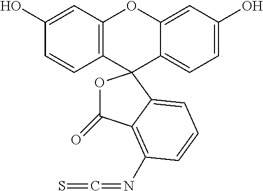 [SMILES: O=C1OC2(c3ccc(O)cc3Oc3cc(O)ccc32)c2cccc(N=C=S)c21]

Methods for forming radiopharmaceuticals by complexing a radioactive isotope, such as ¹⁷⁷Lu or ⁶⁸Ga, with a peptide of formula Z-L-X-X1-X2-Asp-X3, such as EVG321 and other, have been previously described. Illustrative radiolabeling methods are described in, for example, US 2022/0041649, US 2021/0138904, US 2020/0138985, and 2021/0316019.

An illustrative procedure for complexing EVG321 with ¹⁷⁷Lu involves forming a precursor solution by dissolving approximately 200 μg of EVG321 in about 250 μL of a 0.4 M acetate buffer solution (pH 4.0-8.0) containing one or more stabilizers (e.g. ascorbic acid, gentisic acid, etc.), in this case 10 mg of gentisic acid. The precursor solution is then mixed with non-carrier-added ¹⁷⁷LuCl₃ (e.g. 7.4 GBq in approximately 200 μL of 0.04 N HCl) and incubated at about 95° C. for about 20 minutes. The resulting solution is then transferred with 4.5 mL of a 1% aqueous acetic acid solution to a vial containing a solution of additional stabilizers or additives (45 mg ascorbic acid, 0.8 mg DTPA, 11.3 mg NaOH, and 0.9 mL ethanol in 14 mL water) to provide a safe parenteral drug solution. The resulting parenteral drug solution is then mixed and passed through a sterile filter to provide a finished drug product. Typically, aliquots of the finished drug product are distributed for quality control testing, including, for example, analysis for EVG321, radiochemical identity, radiochemical purity, and identity and amount of stabilizers and excipients, before the finished drug product is administered to the patient.

An illustrative procedure for complexing EVG321 with ⁶⁸Ga involves taking a fresh gallium-68 chloride eluate from a ⁶⁸Ge/⁶⁸Ga-generator (˜1000 MBq) and mixing the eluate with a solution of 50 μg EVG321 in a 1.45 M aqueous sodium acetate buffer solution (pH 4.0) that contains 20% (v/v) ethanol. The resulting solution is then heated at about 95° C. for about 7-10 minutes. The resulting heated solution is then loaded onto a pre-treated Sep-Pak® Light C18 solid-phase extraction (“SPE”) cartridge (pre-treated with 50% ethanol/saline) and the radiopharmaceutical eluted using about 1 mL of 50% ethanol followed by about 7.5 mL of saline to yield the finished drug product. Quality control testing of the finished drug product including, for example, appearance, pH, filter integrity, HPLC analysis, TLC analysis, GC analysis, radionuclide identification, and sterility, is performed before the finished drug product is administered to the patient.

The aim of the method is to target binding of the radionuclide to tumor cells while avoiding or minimizing undesirable binding of the radionuclide to other tissues in the body, specifically, the stomach. Without wishing to be bound by theory, it is believed that orally administering the blocking agent prevents binding of the radionuclide to stomach tissue, tissue that has a high concentration of CCK2 receptors.

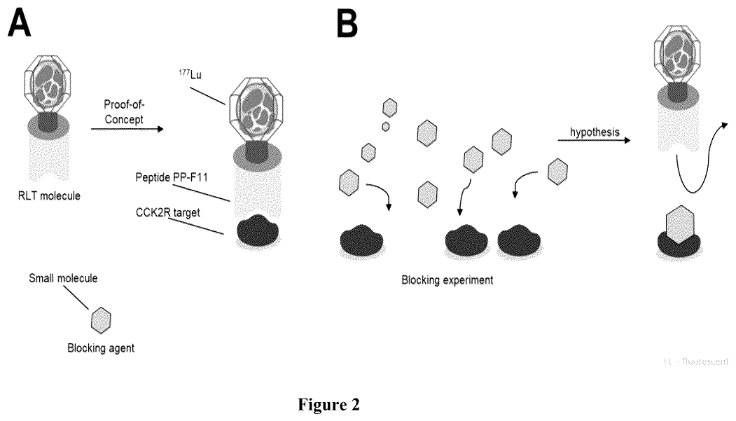

FIG. 2 is a graphical depiction of the proposed mechanism by which the method is believed to work by blocking of EVG321 to receptors in the stomach. FIG. 2A depicts the expected binding of EVG321, where a ¹⁷⁷Lu radiolabeled peptide binds to targets expressing the CCK2 receptor in the stomach. FIG. 2B depicts the blocking agent binding to the CCK2 receptor and preventing binding of EVG321.

It is believed that oral administration of the blocking agent saturates CCK2 receptors in the stomach and prevents binding of the intravenously administered radionuclide without preventing binding of the intravenously administered radionuclide at the tumor. It is believed that the blocking agent saturates CCK2 receptors in the stomach of the patient within about 10 to about 60 minutes following administration. By preventing binding of the radionuclide to CCK2 receptors in the stomach, oral administration of the blocking agent avoids subjecting healthy cells in the stomach to unnecessary and destructive radiation due to undesired binding of the radionuclide and prevents or minimizes radiotoxicities and radiation-induced cellular and sub-cellular damage, which can lead to tissue damage and downstream toxicities (including, but not limited to nausea, vomiting, dyspepsia, and abdominal pain).

The method of treatment is preferably used in patients that exhibit uptake of the radionuclide by CCK2 receptors in the stomach. Patients can exhibit variability in stomach uptake of a radionuclide. Uptake of a radionuclide by CCK2 receptors in the stomach can be demonstrated by evaluating uptake of PP-F11 and EVG321 in vitro as described below in Example 1 and Example 2.

### EXAMPLES

### Example 1

Small molecule antagonism of PP-F11 in AR42J and Panc1 cell lines was evaluated by measuring the intensity of a fluorescent (“FL”) signal of FL tagged PP-F11 in the presence or absence of small molecule inhibitors. PP-F11 targets CCK2 receptor in the same way as EVG321 does (see M. Klingler et al., Site-specific stabilization of minigastrin analogs against enzymatic degradation for enhanced cholecystokinin-2 receptor targeting, Theranostics, 8 (11): 2896-2908 Apr. 16, 2018 and erratum in Theranostics, 9 (16): 4595-4596 Jun. 19, 2019). PP-F11 is also known as CP04 or PP11-D. An isothiocyanate to amine coupling protocol was used to attach FITC to the N-terminus of PP-F11 yielding a fluorescent peptide (also referred to as FITC-PP-F11, PP-F11-NH-FITC, PP-F11-FITC, or FL-PP-F11) followed by HPLC purification.

The chemical structure of PP-F11 is:

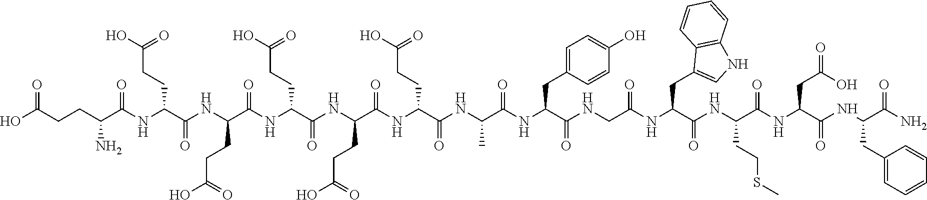 [SMILES: CSCC[C@H](NC(=O)[C@H](Cc1c[nH]c2ccccc12)NC(=O)CNC(=O)[C@H](Cc1ccc(O)cc1)NC(=O)[C@H](C)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)[C@H](N)CCC(=O)O)C(=O)N[C@@H](CC(=O)O)C(=O)N[C@@H](Cc1ccccc1)C(N)=O]

The chemical structure of PP-F11-FITC is:

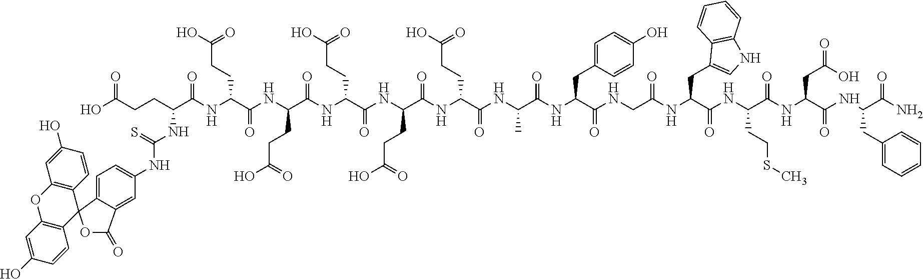 [SMILES: CSCC[C@H](NC(=O)[C@H](Cc1c[nH]c2ccccc12)NC(=O)CNC(=O)[C@H](Cc1ccc(O)cc1)NC(=O)[C@H](C)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)[C@@H](CCC(=O)O)NC(=S)Nc1ccc2c(c1)C(=O)OC21c2ccc(O)cc2Oc2cc(O)ccc21)C(=O)N[C@@H](CC(=O)O)C(=O)N[C@@H](Cc1ccccc1)C(N)=O]

PANC-1 human pancreatic cancer cell line were cultured in Dulbecco's Modified Eagle's Medium (DMEM) medium from ATCC with a final concentration of fetal bovine serum (FBS) of 10% (v/v). Medium was changed every 2-3 days and a sub-cultivation ratio of 1:4 was used to split the cells. For the cryoconservation of the PANC-1 cancer cell line, at passage cycle 3, growth medium DMEM was supplemented with 10% (v/v) FBS and 10% (v/v) sterile DMSO and the resulting PANC-1 cell solution (1.5 mL per vial) were stored under liquid nitrogen for long term storage.

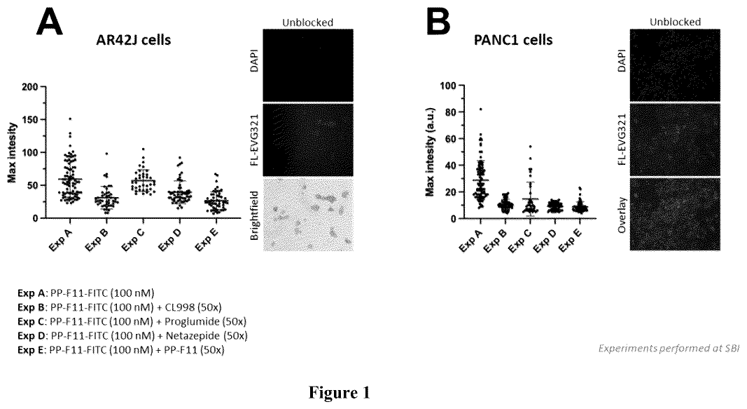

AR42J rat pancreatic cancer cell lines were cultured in F-12K medium from ATCC with a final FBS concentration of 20% (v/v). Medium was changed every 2-3 days and a sub-cultivation ratio of 1:3 was used to split the cells. For the cryoconservation of the AR42J rat cancer cell line, at passage cycle 3, growth medium DMEM was supplemented with 10% (v/v) FBS and 10% (v/v) sterile DMSO and the resulting AR42J cell solution (1.5 mL per vial) was stored under liquid nitrogen for long term storage. FIG. 1A represents experiments in AR42J cells and FIG. 1B represents experiments in Panc1 cells. Each column represents a specific experimental condition. A=PP-F11-FITC, B=FL-PP-F11-FITC+CI988, C=PP-F11-FITC+Proglumide, D=PP-F11-FITC+netrazepide, and E=PP-F11-FITC+PP-F11. Each blocking agent was added in a 50× excess dose concentration to saturate available receptors.

FIG. 1A depicts small molecule blocking experiments performed in rat pancreatic AR42J cells, which are known to express CCK2R. 100 nM of a fluorescently labeled version of PP-F11 (PP-F11-FITC) was added to cell culture in the absence of or the presence of various small molecule CCK2 receptor antagonists at 50× the concentration of fluorescently labeled PP-F11. To determine blocking of PP-F11, the fluorescent intensity of fluorescently labeled PP-F11 was measured in each experiment. A decrease in fluorescent intensity compared to a control containing only PP-F11-FITC (experiment A) is indicative of effective blocking. Experiments B, D, and E (using CL998, netrazepide, and an excess dose of non-fluorescent PP-F11, respectively) showed a decrease in fluorescent signal compared to experiment A, indicating successful blocking of PP-F11 binding.

The experiment was repeated in Panc1 cells, another pancreatic cell line that expresses the CCK2 receptor and similar results were observed. The results are provided in FIG. 1B.

### Example 2

AR42J rat pancreatic cell line were used as they express the CCK2 receptor. The AR42J rat pancreatic cancer cell line was cultured in F-12K medium from ATCC with a final concentration of FBS of 20% (v/v). Medium was changed every 2-3 days and a sub-cultivation ratio of 1:3 was used to split the cells. For cryoconservation of the AR42J rat cancer cell line, at passage cycle 3, growth medium DMEM was supplemented with 10% (v/v) FBS and 10% (v/v) sterile DMSO and the resulting PANC-1 cell solution (1.5 mL per vial) were stored under liquid nitrogen for long term storage. 1×10⁶AR42J cells were seeded per well in a 6 well plate in 2 mL of media (DMEM: F12+10% Fetal Bovine Serum (FBS)). Cells were stored at 37° C. in an incubator starting at time 0 for 24-48 hours (depending on cell adherence). On the day of experiment, 1 hour before adding the blocking agent, the medium was changed and replaced with 2 mL of fresh media, and incubated at 37° C. degrees for 1 hour. Once the 1-hour incubation was complete, 4 μL of blocking agent was added to each well to provide a final concentration of 2 mM (i.e., 100 times more concentrated than the radioactive molecule). The plate was gently shaken and incubated at 37° C. for 1 hour. Once incubation was complete, 200 μL of ¹⁷⁷Lu-EVG321 was added to each well (200 fmols per well) and the plates incubated for an additional hour. After incubation, media with excess unbound ¹⁷⁷Lu-EVG321 was removed, the cells gently washed in each well with 500 μL of PBS, and, after discarding the PBS, 500 μL of 1M NaOH was added to each well to lyse the cells. The contents of each well were then triturated to dislodge and lyse cells, and the collectable sample placed in a gamma-counting tube. An extra 500 μL of PBS was added to each well to collect any residual material and the PBS wash fraction was added to the corresponding gamma-counting tube. Samples were then analyzed using a Hidex Gamma counter to measure counts from each tube (gamma peaks of ¹⁷⁷Lu at 113 keV and 208 keV, 5 minutes per tube counting). Once completed, counts were normalized to the initial added activity of ¹⁷⁷Lu-EVG321 to record the relative counts per minute (relative uptake, CPM). Radioactive uptake was measured as a fraction of the added radiation at time 0 and used to calculate relative binding in counts per minute (CPM).

Three blocking agents were tested. CI-988, proglumide, and sograzepide.

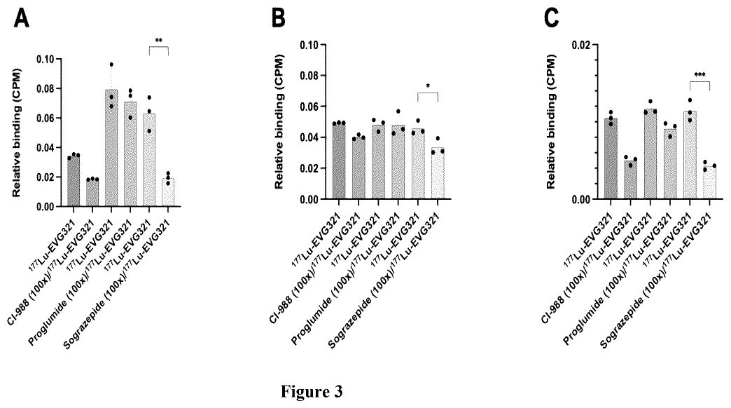

Results: A significant decrease in the specific uptake of ¹⁷⁷Lu-EVG321 was demonstrated when cells were pre-treated with 2 mM sograzepide (100× excess dose) for one hour prior to the experiment (p-values of <0.05, <0.01, and <0.001 for repeat 1, 2, and 3, respectively. FIGS. 3A, B, and C). The blocking agent CI-988 (2 mM CI-988) was able to significantly reduce uptake of ¹⁷⁷Lu-EVG321, while proglumide (2 mM proglumide) did not show as drastic and statistically significant reduction of uptake (FIG. 3A-C). Statistical analysis was performed via a two-tailed t-test where a minimum p<0.05 was considered statistically significant.

What is claimed:

1. A method for diagnosing or treating a patient with a tumor that expresses CCK2 receptors comprising (i) orally administering to the patient a blocking agent and (ii) subsequently intravenously or topically administering to the patient a radiopharmaceutical that binds to a CCK2 receptor.

2. The method of claim 1, wherein the radiopharmaceutical that binds to a CCK2 receptor is of formula:
Z-L-X-X1-X2-Asp-X3
wherein
Z is a chelator capable of chelating a radioactive isotope,
L is absent or is a linker,
X is absent or is an amino acid sequence, and
X1, X2, and X3 are lipophilic amino acids, and
wherein a radioactive isotope is complexed by the chelator.

3. The method of claim 2, wherein X1 is selected from the group consisting of Trp, 3-(1-naphtyl) alanine, 3-(2-naphtyl) alanine, and L-alpha-methyltryptophane, X2 is selected from the group consisting of Met, phenylglycine, isoleucine, N-methylmethionine, N-methylphenylglycine and N-methylisoleucine, and X3 is Phe-NH₂, 3-(1-naphtyl) alanyl amide, 3-(2-naphtyl) alanyl amide, N-methylphenylalanyl amide, N-methyl-3-(1-naphtyl) alanyl amide and N-methyl-3-(2-naphtyl) alanyl amide.

4. The method of claim 2, wherein the radiopharmaceutical that binds to a CCK2 receptor is selected from the group consisting of PP-F11N, DOTA-MGS5, and CCK-66.

5. The method of claim 4, wherein the radioactive isotope is selected from the group consisting of ¹⁸F, ⁶⁴Cu, ⁶⁷Cu, ⁶⁸Ga, ⁸⁹Zr, ⁹⁰Y, ¹²³I, ¹²⁴I, ¹³¹I, ¹⁶¹Tb, ¹⁷⁷Lu, ²⁰³Pb, ²¹¹At, ²¹²Bi, ²¹²Pb, ²¹³Bi, ²²⁵Ac.

6. The method of claim 1, wherein tumor that expresses CCK2 receptors is selected from the group consisting of small cell lung cancer (SCLC) tumors and (medullary thyroid cancer (MTC) tumors.

7. The method of claim 1, wherein the blocking agent is selected from the group consisting of sograzepide, proglumide, lorglumide, devazepide, CI-988, CI-1015, L-365,260, L-369,293, YF-476, RP-69758, LY-288,513, PD-145,942 and L-740,093.

8. The method of claim 7, wherein the blocking agent is sograzepide.

9. The method of claim 8, wherein the sograzepide is administered at a dose ranging from about 20 mg to about 250 mg.

10. The method of claim 7, wherein the blocking agent is CI-988.

11. The method of claim 10, wherein the CI-988 is administered at a dose ranging from about 5 to about 250 mg.

12. The method of claim 1, wherein the blocking agent is administered between about 1 minute and about 180 minutes prior to intravenous or topical administration of the radiopharmaceutical that binds to a CCK2 receptor.

13. The method of claim 12, wherein the blocking agent is administered between about 10 minutes and about 60 minutes prior to intravenous administration of the radiopharmaceutical.

14. The method of claim 1, wherein the radiopharmaceutical is administered intravenously.

15. The method of claim 1, wherein the radiopharmaceutical is administered topically.

16. The method of claim 1, wherein the radiopharmaceutical that binds to a CCK2 receptor is of formula:
DOTA-D-Glu-Ala-Tyr-Gly-Trp-(N-Me)Nle-Asp-1-Nal-NH² and
the radiopharmaceutical is conjugated to ¹⁷⁷Lu.

17. The method of claim 1, wherein the chelator is selected from the group consisting of derivatives of 1,4,7,10-tetraazacylododecane, 1,4,7-triazacyclononane, 1,4,8,11-tetraazacyclotetradecane, diethylenetriamine, 3,6,9,15-tetraazabicyclo[9.3.1]pentadeca-1 (15), 11,13-triene, and 6-aminoperhydro-1,4-diazepine.

18. The method of claim 2, wherein the chelator is DOTA.

19. The method of claim 5, wherein the radioactive isotope is ¹⁷⁷Lu.
